(19) United States
(12) Patent Application Publication
(10) Pub. No.: US 2024/0168111 A1
(43) Pub. Date: May 23, 2024

(54) THERMAL PACKAGE FOR AN ATOMIC DEVICE

(71) Applicant: FieldLine Inc., Boulder, CO (US)

(72) Inventors: Svenja Knappe, Boulder, CO (US); Collin Coolidge, Erie, CO (US); Orang Alem, Erie, CO (US); Kenneth J. Hughes, Lafayette, CO (US)

(21) Appl. No.: 18/516,489

(22) Filed: Nov. 21, 2023

Related U.S. Application Data
(60) Provisional application No. 63/384,481, filed on Nov. 21, 2022.

(51) Int. Cl. G01R 33/26; G01R 33/00

(52) U.S. Cl. CPC G01R 33/26; G01R 33/0052

(57) ABSTRACT

Various embodiments comprise a thermally packaged atomic device. The thermally packaged atomic device comprises an atomic vapor cell, a heater, and an enclosure. The atomic vapor cell is located within the enclosure. The heater heats the atomic vapor cell. The enclosure is filled with a gas selected for comprising a thermal conductivity less than air. The gas comprising a thermal conductivity less than air may comprise xenon or krypton. The enclosure surrounds the atomic vapor cell with the gas.

### RELATED APPLICATIONS

This U.S. Patent application claims the benefit of and priority to U.S. Provisional Patent Application 63/384,481 titled, “THERMAL PACKAGE FOR AN ATOMIC DEVICE” which was filed on Nov. 21, 2022, and which is hereby incorporated by reference in its entirety into this U.S. Patent Application.

### TECHNICAL FIELD

Various embodiments of the present technology relate to atomic devices, and more specifically, to insulating atomic devices using a thermal package.

### BACKGROUND

Atomic devices use atomic transitions to measure physical quantities, such as time, magnetic fields, or electric fields. Atomic magnetometers or Optically-Pumped Magnetometers (OPMs) detect and characterize magnetic fields of a target. Atomic clocks are precise frequency references. Atomic gyroscopes measure the rotation of a platform. Electrometers measure electric fields generated by a target. Atomic devices include physics packages that comprise optics, light sources, and atomic or molecular material. The physics packages are powered and operated by a controller. The controller processes the signals from the physics package. Inside the physics package, atomic or molecular material like alkali vapor is confined inside a glass vapor cell or oven. The glass vapor cells receive light from the light sources to measure the physical quantity (e.g., the target magnetic field). Metallic alkali vapor is heated to create an optimal vapor pressure within the cell. This requires a substantial amount of power to operate the atomic device. In atomic devices such as OPMs, the heat of the atomic vapor cell can affect the target. For example, in an OPM-based Magnetoencephalography (MEG) system, the OPMs are placed on the head of the subject to detect magnetic fields originating inside the subject's head. Heat generated by heating the vapor cells can become uncomfortable for the subject.

To mitigate unwanted heat transfer, conventional vapor cells are enclosed inside a vacuum. However, the hermetic structures that contain the vacuum are difficult and costly to construct in a way that maintains a low enough pressure in the enclosure over the lifecycle of the device. Loss of vacuum can degrade the performance of the atomic device and increase the power consumption. The vacuum can be degraded from outgassing of material inside the enclosure or leaking of gas through the walls of the enclosure. In atomic devices, often one or several walls of the enclosure are made from a transparent material. Diffusion of gas such as helium through the transparent material can degrade the vacuum and also performance of the atomic device.

Unfortunately, conventional vapor cell heat mitigation enclosures do not effectively and efficiently insulate the vapor cells. Moreover, the conventional vapor cell heat mitigation enclosures are difficult to construct.

### Overview

This Overview is provided to introduce a selection of concepts in a simplified form that are further described below in the Technical Description. This summary is not intended to identify key features or essential features of the claimed subject matter, nor is it intended to be used as an aid in determining the scope of the claimed subject matter.

Various embodiments of the present technology relate to atomic device insulation. Some embodiments comprise a thermally packaged atomic device. The thermally packaged atomic device comprises an atomic vapor cell, at least one heater, and an enclosure. The at least one heater heats the atomic vapor cell. The enclosure is filled with a gas selected for comprising a thermal conductivity less than air. The enclosure surrounds the atomic vapor cell with the gas.

Some embodiments comprise a method of manufacturing a thermally packaged atomic device. The method of manufacturing comprises mounting an atomic vapor cell to an enclosure base. The method further comprises placing enclosure walls on the enclosure base to surround the atomic vapor cell. The method further comprises clamping the enclosure walls to the enclosure base. The method further comprises bonding the enclosure walls to the enclosure base at an interface between the enclosure walls and the enclosure base. The method further comprises backfilling a surrounding environment of the atomic vapor cell with a gas selected for comprising a thermal conductivity less than air and placing an enclosure top on the enclosure walls to create an enclosed volume that surrounds the atomic vapor cell with the gas. The method further comprises clamping the enclosure top to the enclosure walls. The method further comprises bonding the enclosure top to the enclosure walls at an interface between the enclosure top and the enclosure walls.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary thermally packaged atomic device.

FIG. 2 further illustrates the thermally packaged atomic device.

FIG. 3 further illustrates the thermally packaged atomic device.

FIG. 4 illustrates an exemplary thermally packaged atomic device.

FIG. 5 further illustrates the thermally packaged atomic device.

FIG. 6 further illustrates the thermally packaged atomic device.

FIG. 7 further illustrates the thermally packaged atomic device.

FIG. 8 illustrates an exemplary method of manufacturing a thermally packaged atomic device.

FIG. 9 illustrates an exemplary Magnetoencephalography (MEG) system to sense a target magnetic field with thermally packaged Optically Pumped Magnetometers (OMPs).

FIG. 10 illustrates an exemplary operation of the MEG system to sense a target magnetic field with thermally packaged OPMs.

FIG. 11 illustrates an exemplary thermally packaged OPM in the MEG system.

FIG. 12 further illustrates the thermally packaged OPM in the MEG system.

### DETAILED DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

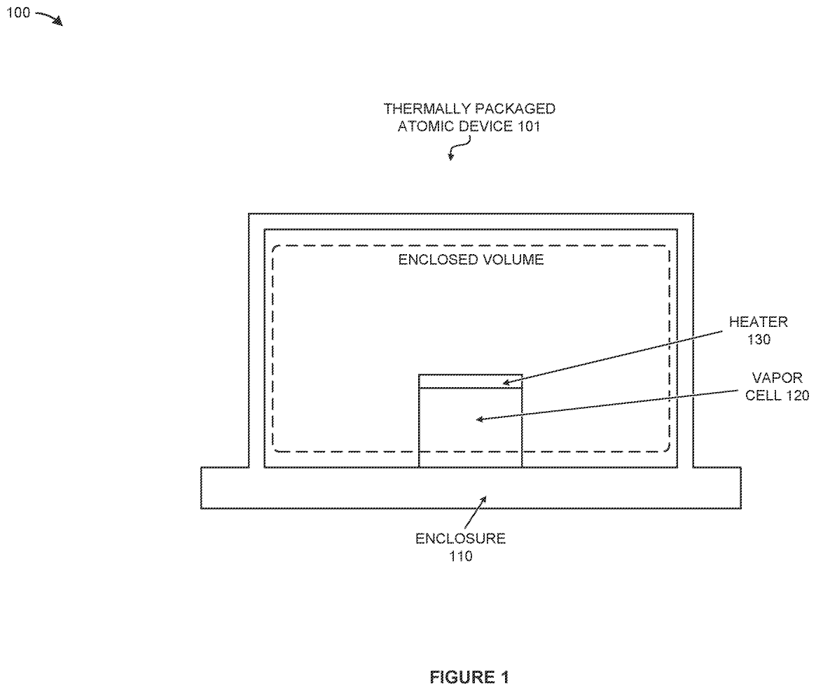

FIG. 1 illustrates view 100. View 100 is representative of a cross-sectional view of thermally packaged atomic device 101. Atomic device 101 comprises enclosure 110, vapor cell 120, and heater 130. The interior of enclosure 110 forms an enclosed volume. The enclosed volume is filled with a gas selected for comprising a thermal conductivity less than that of air at an equal pressure. In other examples, atomic device 101 may comprise additional or different elements than those illustrated in FIG. 1.

Various examples of atomic device configurations and manufacturing operations are described herein. In some examples, atomic vapor cell 120 comprises a hollow construction that holds a vapor. The vapor comprises atoms selected for comprising properties useful for an application. Vapor cell 120 may comprise a vapor cell for magnetometry, electrometry, a gyroscope, a frequency reference, or another type of atomic device. Typically, the type of vapor housed by cell 120 depends in part on the intended function of the atomic device. For example, in magnetic field sensing embodiments, the vapor may comprise rubidium vapor while in atomic frequency reference embodiments, the vapor may comprise cesium vapor. Vapor cells typically comprise windows that allow light to pass through the cell and interact with the atoms that compose the vapor. For example, a laser beam may be passed through cell 120 to probe the atoms. The interaction between the beam and the atoms alters the beam in a way correlated with the physical state of the atoms. The beam may then be detected, and the alteration measured to infer the physical state of the atoms. Heater 130 is attached to the surface of vapor cell 120. Heater 130 heats vapor cell 120 to energize the vapor within cell 120 thereby increasing the pressure of the vapor. The increase in cell pressure facilitates cell probing operations (e.g., by laser beam) to determine the physical state of the atoms.

The enclosed volume formed by enclosure 110 is filled with a gas selected for comprising a thermal conductivity less than air when held at an equal pressure. Thermal conductivity is a measure of the ability of a material to transfer heat through conduction. Materials with a high thermal conductivity (e.g., copper) more readily transfer heat through conduction than materials with a low thermal conductivity (e.g., polystyrene foam). Exemplary gases that comprise a thermal conductivity lower than air (at the same temperature) include argon, xenon, and krypton. The filling gas in enclosure 110 insulates vapor cell 120 from the external environment by inhibiting conductive heat transfer between vapor cell 120 and the interior surfaces of enclosure 110. The insulation inhibits heat loss from vapor cell 120 to increase the operational efficiency of vapor cell 120 and reduce the power requirements to heat vapor cell 120.

The thermal conductivity of a gas depends in part on the particle size and temperature of the gas. Gas thermal conductivity decreases as particle size increases and increases as temperature increases. The filling gas is also selected for comprising physical stability and low to no reactivity with the other materials of the atomic device. For example, although radon comprises a lower thermal conductivity than air, radon may not be a suitable filling gas because of its low atomic stability due to radioactive decay. Similarly, although fluorine has a lower thermal conductivity than air, fluorine may not be a suitable filling gas due to its high reactivity which can damage the other components within the enclosed volume. As such, the filling gas of atomic device 101 is both chemically stable and inert with respect to the other components of device 101. For example, argon, xenon, and krypton are exemplary filling gases due to their lower thermal conductivity than air, non-reactivity with the other components of atomic device 101, and their atomic stability (e.g., non-radioactive).

Advantageously, the thermal packaging of atomic device 101 effectively and efficiently insulates atomic vapor cell 120. Moreover, filling the enclosed volume of atomic device 101 with a gas comprising a thermal conductivity lower than air allows the atomic device to be efficiently constructed when compared to conventional vacuum enclosures. Filling the enclosure with a gas like argon, krypton, or xenon rather than evacuating it simplifies the manufacturing process and reduces the hermiticity requirement of enclosure 110. Furthermore, filling enclosure 110 with a gas like argon, krypton, or xenon reduces thermal conductivity when compared to an air-filled enclosure.

Vapor cell 120 may comprise a glass vapor cell, a silicon-glass vapor cell, or some other type of vapor cell. The vapor housed by cell 120 may comprise alkali vapors like rubidium vapor, cesium vapor, or another type of vapor. Heater 120 may comprise a metallic thin-film deposited on the surface of vapor cell 120 or another type of resistive heat element. Enclosure 110 may comprise silicon, glass, hermetic seals, adhesives, and the like. For example, the base of enclosure may comprise a silicon wafer while the walls and top of enclosure 110 may comprise glass attached to the base by an adhesive. The insulating gas that fills the interior of enclosure 110 may comprise argon, krypton, xenon, and the like.

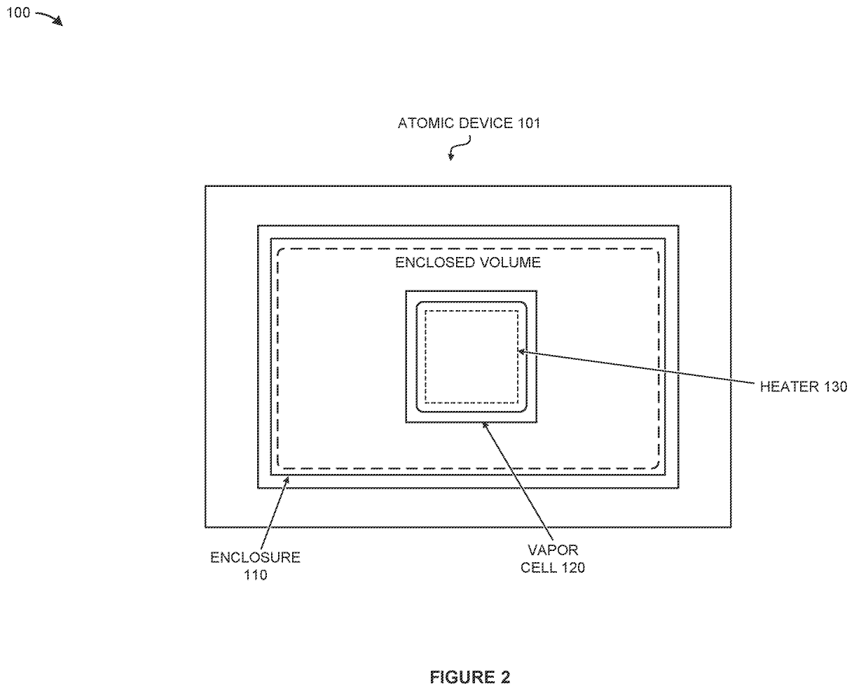

FIG. 2 illustrates view 200. View 200 is representative of a top-down cross-sectional view of atomic device 101. View 200 comprises atomic device 101, enclosure 110, vapor cell 120, and heater 130. The top of enclosure 110 is omitted for clarity.

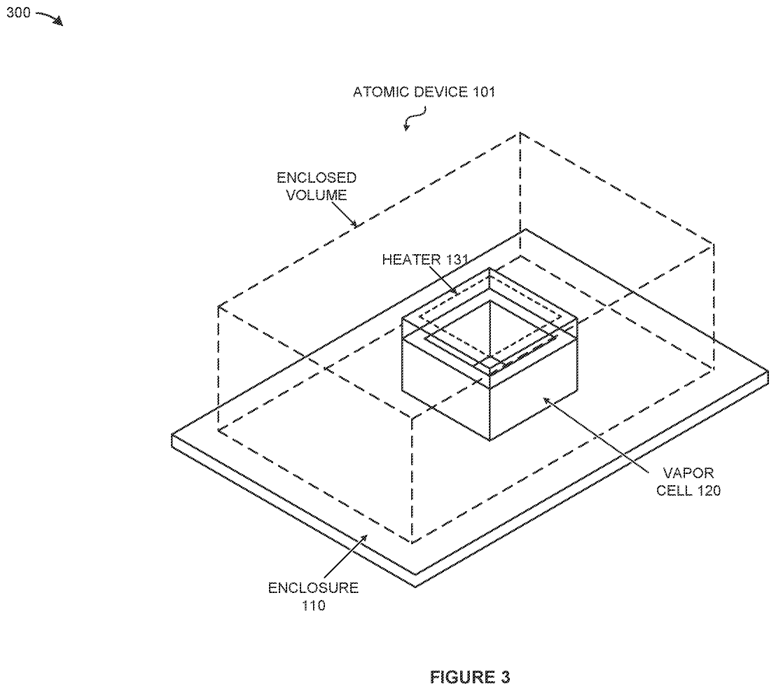

FIG. 3 illustrates view 300. View 300 is representative of a perspective view of atomic device 101. View 300 comprises atomic device 101, enclosure 110, vapor cell 120, and heater 130. The walls and top of enclosure 110 are omitted for clarity.

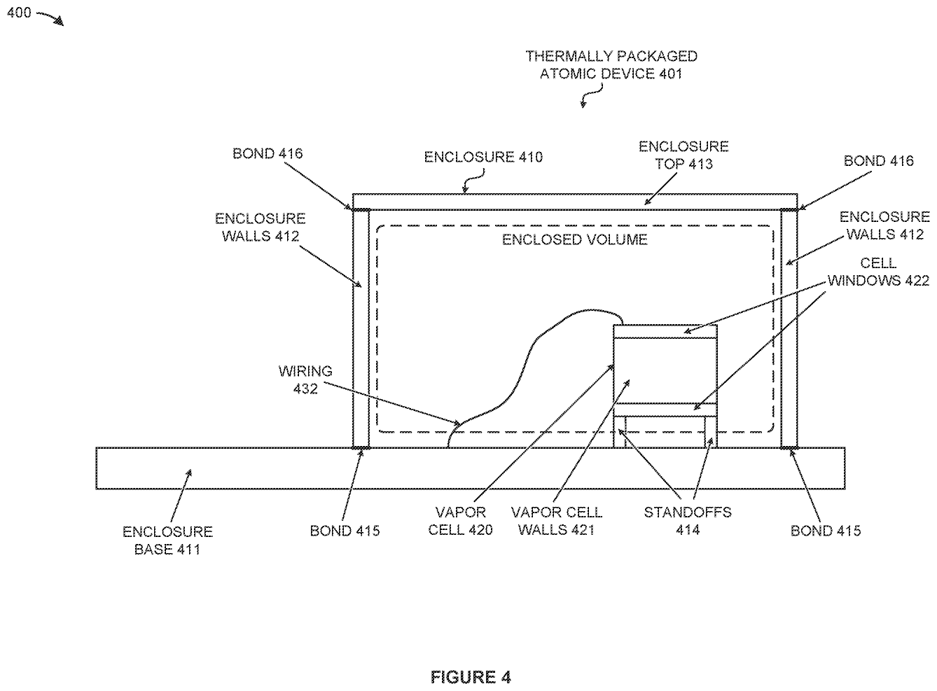

FIG. 4 illustrates view 400. View 400 is representative of a cross sectional view of thermally packaged atomic device 401. Atomic device 401 comprises an example of device 101 illustrated in FIGS. 1-3, however device 101 may differ. Atomic device 401 comprises enclosure 410, vapor cell 420, heater 431, wiring 432, and electrical feedthroughs 433. Enclosure 410 comprises enclosure base 411, enclosure walls 412, enclosure top 413, standoffs 414, bond 415, and bond 416. Vapor cell 420 comprises vapor cell walls 421, cell windows 422, and cell cavity 423. Enclosure base 411, walls 412, and top 413 form an enclosed volume. The enclosed volume is filled with a gas selected for comprising a thermal conductivity less than that of air at an equal pressure. In other examples, atomic device 401 may comprise additional or different elements than those illustrated in FIG. 4.

In some examples, enclosure walls 412 are attached to enclosure base 411 by bond 415 to create a hermetic seal. Similarly, enclosure top 413 is attached to enclosure walls 412 by bond 416 to create a hermetic seal. Bond 415 may comprise epoxy, soldering, anodic bonds, laser welds, laser bonds, thermocompression bonds, direct bonds, and/or another suitable bond type. Bond 416 may comprise epoxy, soldering, anodic bonds, laser welds, laser bonds, thermocompression bonds, direct bonds, and/or another suitable bond type. Bonds 415 and 416 may comprise combinations of bond types. For example, bond 416 (i.e., between top 413 and walls 412) may comprise a solder bond and a laser weld. Typically, the bond type is selected to hermetically seal the enclosed volume to inhibit gas exchange between the enclosed volume and the exterior environment and to inhibit outgassing of the bonding agent into the enclosed volume. For example, an epoxy that exhibits outgassing above a threshold may not be suitable to create the hermetic seal.

Enclosure base 411 may comprise a silicon printed circuitry board, a glass piece with deposited circuitry, and/or other types of insulating materials that inhibit gas diffusion and that support embedded circuitry. For example, enclosure base 411 may comprise a printed circuit board bonded that is soldered and laser welded to enclosure walls 412. Enclosure walls 412 may comprise glass, borosilicate glass, aluminosilicate glass, 3D printed plastic, silicon and/or other types of insulating materials that inhibit gas diffusion. For example, enclosure walls 412 may comprise an aluminosilicate glass that inhibits helium diffusion through its surfaces. Enclosure top 413 may comprise silicon, glass, borosilicate glass, aluminosilicate glass, 3D printed plastic, and/or other types of insulating materials that inhibit gas diffusion. For example, enclosure top 413 may comprise a silicon piece that is soldered and laser welded to enclosure walls 412.

Vapor cell 420 is mounted to enclosure base 411 within the enclosed volume by standoffs 414. Standoffs 414 comprise insulating materials like glass, polyimide, foam, insulation, and/or plastic. Standoffs 414 comprise posts that elevate the vapor cell above the surface of the enclosure base to reduce surface area contact between cell 410 and base 411. The reduced surface contact reduces conductive heat transfer between the surface of vapor cell 420 and enclosure base 411. Standoffs 414 may mechanically couple to vapor cell 420 by male/female sockets, male/female screw ports, male/female clips, and the like. Alternatively, standoffs 414 may adhere to vapor cell 420 by an adhesive like an epoxy or solder. Standoffs 414 couple to enclosure base 411 mechanically, chemically, and/or through a wafer bonding technique like laser welds. In some examples, standoffs 414 may be replaced by a glass bead, a 3D printed vapor cell mount, and/or another type of mounting device that secures vapor cell 420 to base 411 and that inhibits conductive heat transfer between vapor cell 420 and enclosure base 411.

Vapor cell 420 houses a vapor selected for the function of atomic device 401. Vapor cell walls 421 and cell windows 422 form cell cavity 423 that houses the vapor. Windows 422 are positioned on two opposing sides of cell 410. Vapor cell windows 422 comprise glass pieces that seal the ends of vapor cell cavity 423. Vapor cell cavity 423 comprises a hollow region within vapor cell 420 where the vapor is located. Windows 422 allow a laser beam to pass through cell cavity 423 to probe the vapor housed by cell 420. The beam can then be measured to determine a physical property of the atoms that compose the vapor. Vapor cell 420 may comprise a vapor cell for magnetometry, electrometry, a gyroscope, a frequency reference, or another type of atomic device. The volume enclosed by enclosure 410 is filled with a gas that comprises a thermal conductivity less than air when held at an equal pressure. The gas may comprise xenon, krypton, and the like. The filling gas inhibits conductive heat transfer between vapor cell 420 and enclosure top 413, enclosure walls 412, and enclosure base 411. The insulation reduces heat loss from vapor cell 420 thereby reducing the power requirement of heater 431 to maintain the cell pressure of cell 420. In this example, the gas pressure within the enclosed volume is around 400 kPa, however other gas pressures may be used.

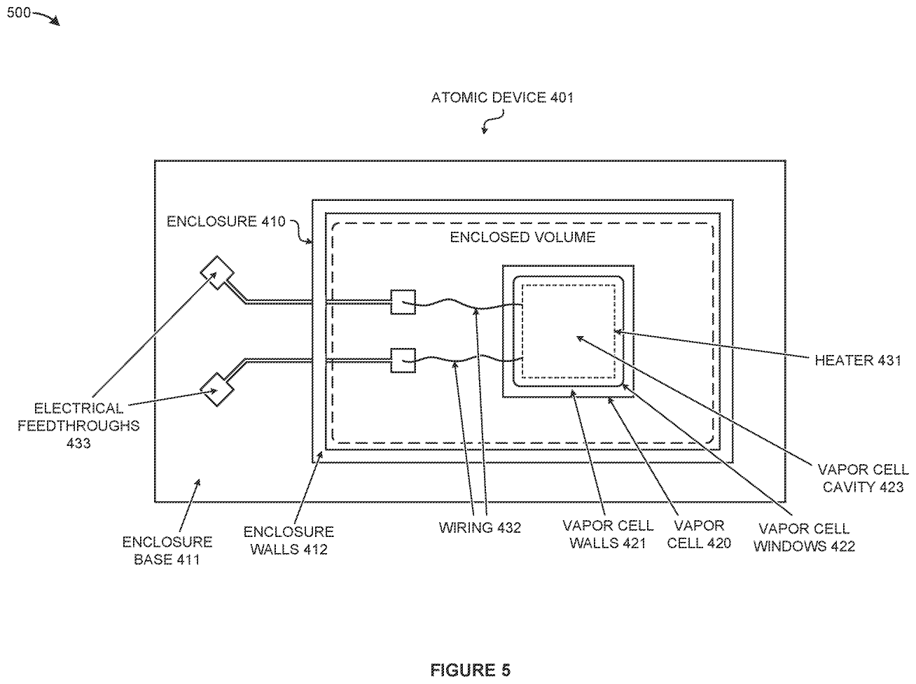

FIG. 5 illustrates view 500. View 500 is representative of a top-down cross view of the atomic device with thermal packaging illustrated in FIG. 4. View 500 comprises atomic device 401, enclosure 410, enclosure base 411, enclosure walls 412, vapor cell 420, vapor cell walls 421, vapor cell window 422, vapor cell cavity 423, heater 431, wiring 432, and electrical feedthroughs 433.

Heater 431 comprises a resistive heating element that heats the vapor housed by cell 420 to maintain cell pressure during operation. For example, heater 431 may comprise wiring embedded into the surface of one of windows 422. Wiring 432 connects heater 431 mounted to the surface of vapor cell 420 to electrical feedthroughs 433 which are embedded to the surface of enclosure base 411. Wiring 432 may comprise sheathed or unsheathed metallic wiring. Wiring 432 delivers current to heater 431 to heat vapor cell 420. In some examples, heater 431 may be omitted and the heat may instead be supplied by an optical source like a laser. Electrical feedthroughs 433 comprise transmission lines to carry electric current from the exterior environment into the enclosed volume to power the operation of heaters 431. In some examples, electrical feedthroughs 433 may comprise bus circuitry on a printed circuit board. In some examples, electrical feedthroughs 433 may comprise a thin film deposition on glass. The thin film deposition may comprise gold, copper, and the like. The interior facing ends of electrical feedthroughs 433 are coupled to wiring 432. The exterior facing ends of electrical feedthroughs 433 may be coupled to a power source (e.g., a battery). Although illustrated as a planar trace on the surface of enclosure base 411, in other examples electrical feedthroughs 433 may go through base 411. For example, base 411 may comprise a printed circuit board and electrical feedthroughs 433 may comprise circuitry of the printed circuit board.

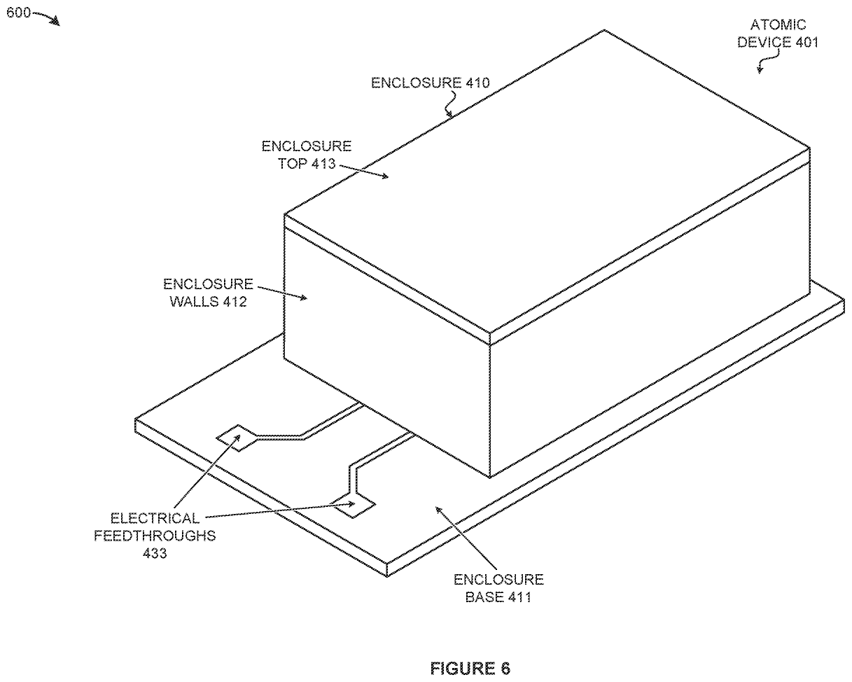

FIG. 6 illustrates view 600. View 600 is representative of an external perspective view of atomic device 401. View 600 comprises atomic device 401, enclosure 410, enclosure base 411, enclosure walls 412, enclosure top 413, and electrical feedthroughs 433. The components positioned within the enclosed volume formed by enclosure walls 412, enclosure base 411, and enclosure top 413 are omitted for the sake of clarity.

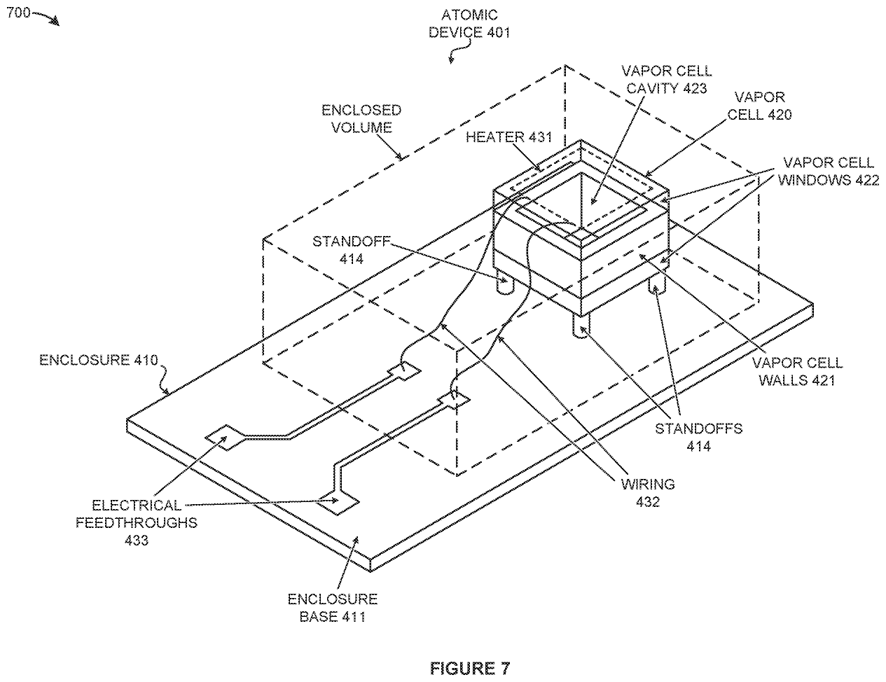

FIG. 7 illustrates view 700. View 700 is representative of an internal perspective view of atomic device 401. View 700 comprises atomic device 401, enclosure 410, enclosure base 411, standoffs 414, vapor cell 420, vapor cell walls 421, vapor cell window 422, vapor cell cavity 423, heater 431, wiring 432, and electrical feedthroughs 433. Enclosure walls 412 and enclosure top 413 are omitted for the sake of clarity. Standoffs 414, vapor cell 420, vapor cell walls 421, vapor cell window 422, vapor cell cavity 423, heater 431, and wiring 432 are positioned within the enclosed volume. The enclosed volume is filled with a gas selected for comprising a thermal conductivity less than air (e.g., xenon or krypton) when held at the same pressure.

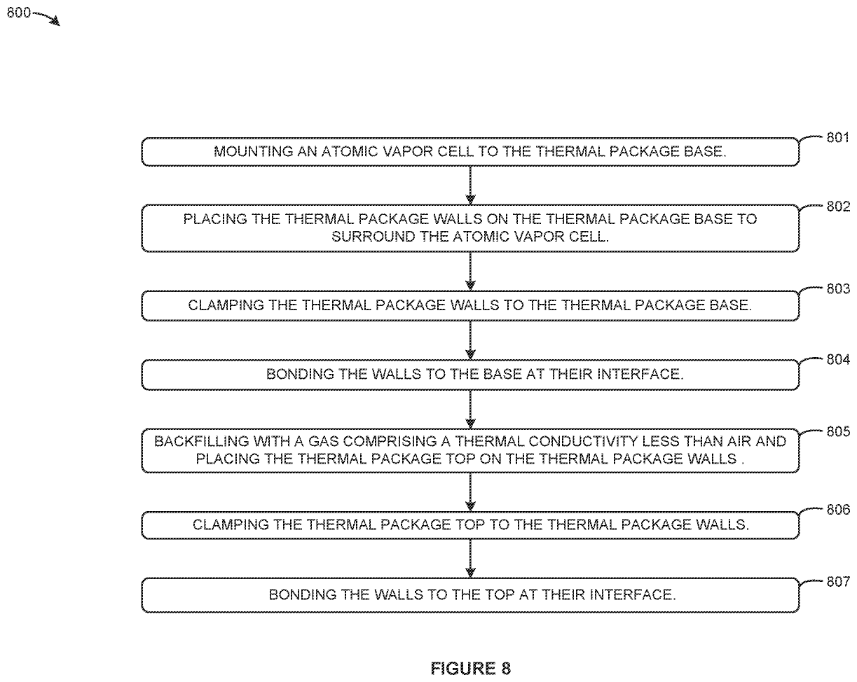

FIG. 8 illustrates process 800. Process 800 comprises an exemplary method of manufacturing thermally packaged atomic devices. The operations of process 800 may differ in other examples. The operations comprise mounting an atomic vapor cell to the thermal package base (step 801). The operations further comprise placing the thermal package walls on the thermal package base to surround the atomic vapor cell (step 802). The operations further comprise clamping the thermal package walls to the thermal package base (step 803). The operations further comprise bonding the walls to the base at their interface (step 804). The operations further comprise backfilling with a gas comprising a thermal conductivity less than air and placing the thermal package top on the thermal package walls (step 805). The operations further comprise clamping the thermal package top to the thermal package walls (step 806). The operations further comprise bonding the walls to the top at their interface (step 807). Referring back to FIGS. 4-7, views 400-700 include a brief example of process 800 to manufacture thermally packaged atomic device 401. The operation may differ in other examples.

In operation, standoffs 414 are attached to enclosure base 411. Standoffs 414 may attach to base 411 using solder, glue, snap connectors, and the like. Standoffs 414 are positioned on base 411 along its lengthwise central axis. Standoffs 414 are spaced apart from each other to align with the outer dimensions of vapor cell 420. For example, if the bottom face of cell 420 comprises a square with a surface area of 0.5 cm×0.5 cm, standoffs 414 may be arranged on base 411 to align with the vertices of the bottom face of cell 420. Once attached to base 411, an adhesive or solder is applied to the exposed (e.g., upper) surfaces of standoffs 414. Vapor cell 420 is then placed on the exposed surfaces of standoffs 414. The adhesive/solder binds cell 420 to standoffs 414 to mount cell 420 to base 411 (step 801). Wiring 432 is soldered to contact points on heater 431 and electrical feedthroughs 433.

Enclosure walls 412 are placed onto base 411 to surround vapor cell 420 (step 802). Walls 412 are arranged as a rectangle with vapor cell 420 located within the perimeter of the rectangle. Walls 412 are clamped to base 411 to secure walls 412 to base 411 during the bonding operation (step 803). The clamping force may be achieved through physical action (e.g., a metal clamp) or through electrostatic force. Electrostatic force may be used when walls 412 comprise glass (e.g., borosilicate glass) and base 411 comprises silicon. In such cases, an electric potential is applied across walls 412 and base 411. The electric potential causes the surfaces of walls 412 and base 411 to develop opposing charges that electrostatically bind walls 412 to base 411.

Once walls 412 are securely clamped to base 411, walls 412 are bonded to base 411 to create a hermetic seal (step 804). In this example, a laser welding tool is used to weld walls 412 to base 411 to create bond 415. In other examples, base 411 may be attached to walls 412 using other (or additional) bonding techniques like anodic bonding, soldering, an adhesive, or another type of hermetic bond. For example, walls 412 may be laser welded to base 411 and then a solder may be applied at their interface increase the hermiticity of enclosure 410. After bonding is complete, the volume surrounding vapor cell is backfilled with a gas comprising a lower thermal conductivity than air while enclosure top 413 is placed on walls 412 (step 805). In this example, the gas comprises xenon. For example, a spray nozzle may shoot a jet of xenon gas into the volume that will be enclosed by top 413. The xenon jet displaces the air surrounding cell 420. When top 413 contacts walls 412, the enclosed volume formed by enclosure 410 is filled with the xenon. It should be appreciated that since the air proximate to cell 420 was not evacuated (e.g., by a vacuum), trace amounts of air will be in the enclosed volume, however the majority of the gas in the enclosed volume comprises xenon. In other examples, enclosure 410 may be backfilled with another gas (e.g., krypton). In should be appreciated that since the air is not evacuated prior to backfilling with xenon, a manufacturing process of atomic device 401 is simplified.

Enclosure top 413 is clamped to walls 412 to seal the xenon within the enclosed volume and to secure top 413 to walls 412 during bonding (step 806). Once top 413 is clamped to walls 412, top 413 is bonded to walls 412 to create a hermetic seal. In this example, a laser welding tool is used to weld top 413 to walls 412 to create bond 416. In other examples, top 413 may be attached to walls 412 using other (or additional) bonding techniques like anodic bonding, soldering, an adhesive, or another type of hermetic bond. In other examples, process 800 may include steps to evacuate the air in the environment surrounding vapor cell 420. For example, a vacuum may be pulled to remove the air surrounding cell 420 and then xenon or krypton may be sprayed into the region that will form the enclosed volume. Although this increases the complexity of the manufacturing processes, the purity of the filling gas is increased.

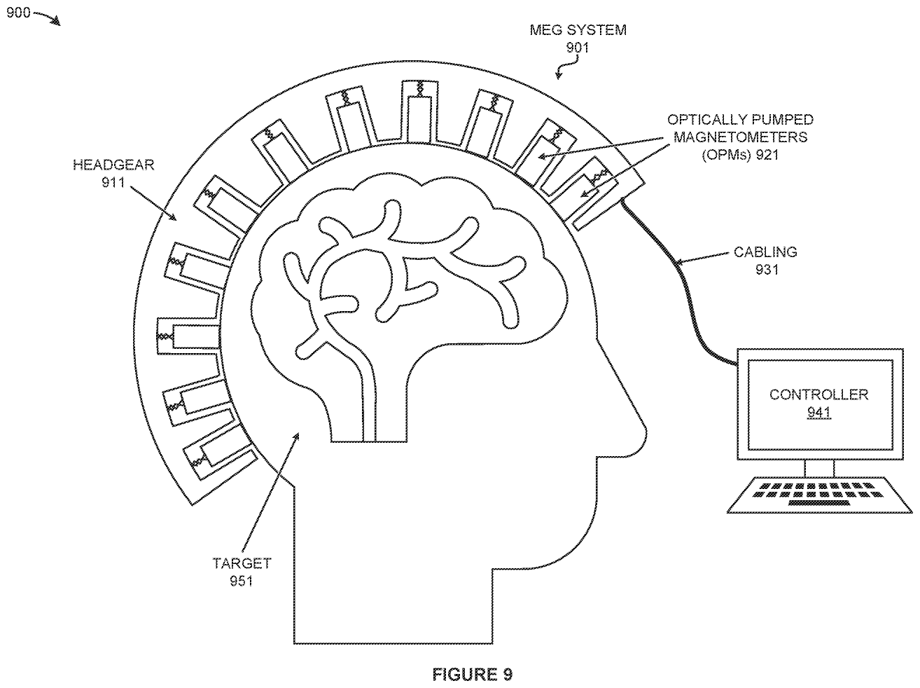

FIG. 9 illustrates view 900. View 900 is representative of MEG system 901. MEG system 901 performs operations like detecting magnetic fields and relating the detecting magnetic fields to neuronal activity for use in medical applications. Exemplary medical applications include identifying brain activity and diagnosing medical conditions like stroke, epilepsy, neuronal injuries, neuronal disorders, and/or other types of medical conditions relating to brain/neuron activity. MEG system 901 comprises headgear 911, Optically Pumped Magnetometers (OPMs) 921, cabling 931, controller 941, and target 951. Target 951 is depicted as a human brain but may comprise any magnetic field source. In other examples, MEG system 901 may comprise additional or different elements than those illustrated in FIG. 9.

Headgear 911 mounts OPMs 921 at spatial locations proximate to the head of target 951. Headgear 911 is shaped to conform to the human head. Headgear 911 may comprise a rigid helmet, a flexible cap, or some other type of headgear configured to hold the sensors. OPMs 921 are coupled to headgear 911. When headgear 911 is worn by target 951, OPMs 921 contact the scalp of target 951. OPMs 921 are an example of a magnetometer. OPMs 921 detect magnetic fields generated by target 951 and transfer signaling characterizing the magnetic field to controller 941. OPMs 921 comprise components like thermal packaging, lasers, coils, vapor cells, photo detectors, and heaters. The thermal packaging houses the vapor cells and heaters to inhibit heat transfer from the heaters to the external environment. Cabling 931 comprises sheathed metallic wiring that communicatively couples OPMs 921 to controller 941. Controller 941 comprises a computing device that transfers instructions to OPMs 921 to drive the operation of MEG system 901 and processes magnetic field data received from OPMs 921. Controller 941 comprises components like processors, memory, transceivers, bus circuitry, and the like. The memory stores software like operating systems, magnetic field modeling applications, control modules, user interface applications, and the like. The processors retrieve and execute the software from the memory to drive the operation of MEG system 901.

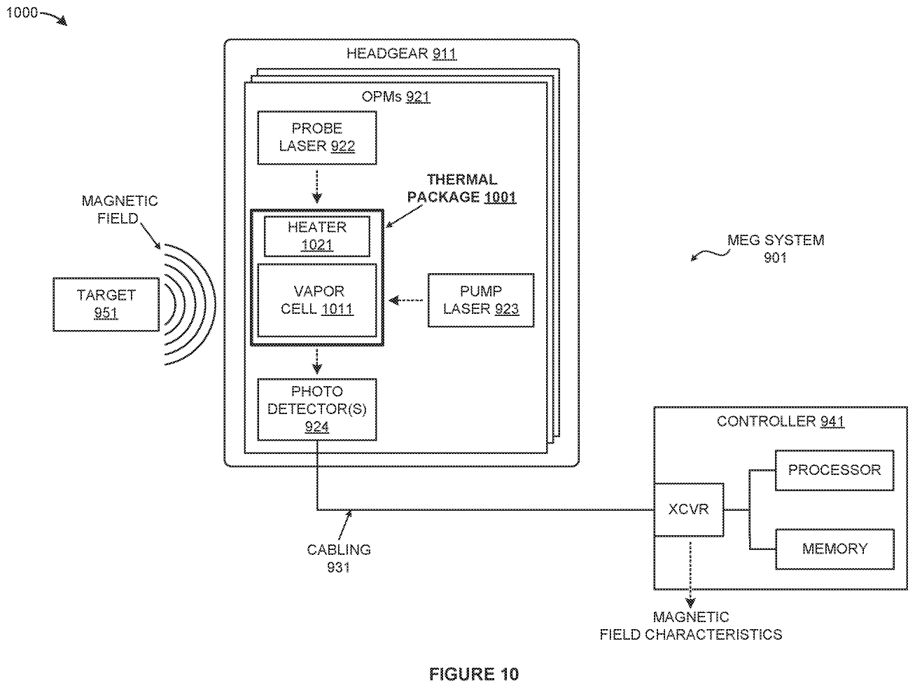

FIG. 10 illustrates view 1000. View 1000 is representative of a component view of MEG system 901. As illustrated in view 1000, MEG system 901 comprises headgear 911, OPMs 921, cabling 931, controller 941, and target 951. Target 951 is magnetically linked to OPMs 921. OPMs 921 are metallically linked to cabling 931 which is metallically linked to controller 941. OPMs 921 each comprise probe laser 922, pump laser 923, photodetectors 924, thermal package 1001, vapor cell 1011, and heater 1021. OPMs 921 typically comprise other components like bias coils, transceiver circuitry, signal processors, flash circuitry, and/or other circuitry, however these additional components are omitted for clarity. In some examples, the two lasers may be combined and/or additional lasers may be used. Thermal package 1001 comprises an enclosed volume that houses vapor cell 1011 and heaters 1021. The enclosed volume is filled with xenon, krypton, or another gas that comprises a lower thermal conductivity than air when held at the same pressure and that does not inhibit the operations of OPMs 921.

Controller 941 comprises transceiver circuitry (XCVR), memory, and a processor. The processor comprises a Central Processing Unit (CPU), Graphics Processing Unit (GPU), Digital Signal Processor (DSP), Field Programmable Gate Array (FPGA), Application Specific Integrated Circuit (ASIC), and/or some other type of processing circuitry. The memory comprises Random Access Memory (RAM), Hard Disk Drive (HDD), Solid Stated Drive (SSD), Non-Volatile Memory Express (NVMe) SSD, and the like. The memories store software like operating systems (OS), and MEG applications for OPM operation, OPM configuration, and magnetic field data processing. The processor retrieves the software from the memory and executes the software to drive the operation of the MEG system 901 as described herein. In the following example, OPMs 921 are referred to in the singular for sake of clarity.

In some examples, headgear 911 is worn by target 951. OPM 921 is adjusted to contact the surface of target 951. For example, headgear 911 may comprise a ratchet mechanism that moves OPM 921 until in contact with target 951. Once in contact with target 951, the processor in controller 941 retrieves and executes an OPM application to generate control signals for OPMs 921. The OPM application selects initial operating parameters like heater temperature, laser wavelength, and bias coil strength based on OPM data stored in the memory. The OPM application generates instructions that direct OPM 921 to measure the magnetic field generated by target 951. The processor drives the transceiver circuitry to transfer the instructions and operating parameters to OPM 921.

Vapor cell 1011 of OPM 921 is positioned in the target magnetic field. Vapor cell 1011 comprises a cell cavity that contains an alkali metal like rubidium. OPM 921 operates in response to the instructions from controller 941. Heaters 1021 heat vapor cell 1011 to vaporize the alkali metal and pressurize vapor cell 1011. Thermal package 1001 inhibits heat transfer from heaters 1021 to the other components of OPM 921. The reduction in heat loss from vapor cell 1011 reduces the power requirement of heater 1021 to maintain the cell pressure of vapor cell 1011. The lowered power requirement increases the operating efficiency of OPM 921. Moreover, the reduced heat loss inhibits the scalp of target 951 from becoming too hot thereby increasing the comfort of target 951.

In this example, OPM 921 comprises a directional magnetometer. The bias coils (not illustrated) in OPM 921 generate a bias magnetic field to orient the sensing direction of OPM 921 to align with the target magnetic field. The sensing direction of an OPM is typically oriented to measure a specific component (e.g., the normal component) of the target magnetic field. Pump laser 923 emits a pump beam that is circularly polarized at a resonant frequency of the vapor contained by vapor cell 1011 to polarize the atoms. Probe laser 922 emits a probe beam that is linearly polarized at a non-resonant frequency of the vapor to probe the atoms. The probe beam enters the vapor cells where quantum interactions with the atoms in the presence of the target magnetic field alter the energy/frequency of probe beam by amounts that correlate to the field strength of the target magnetic field.

Photodetector 924 detects the probe beam after these alterations by the vapor atoms responsive to the magnetic field. Photodetector 924 generates corresponding analog electronic signals that characterize the measured field strength of the magnetic field. In some examples, a signal processor (not illustrated) may filter, amplify, digitize, or perform other tasks on the electronic signals. Photodetector 924 transfers an electronic signal that carries the data over cabling 931 to controller 941. Controller 941 processes the electronic signal received from OPM 921. The processor in controller 941 retrieves and executes a MEG application for magnetic field data processing. The MEG application processes the signal received from OPM 921 to generate data that characterizes the measured field strength (or other magnetic field characteristics) of the target magnetic field.

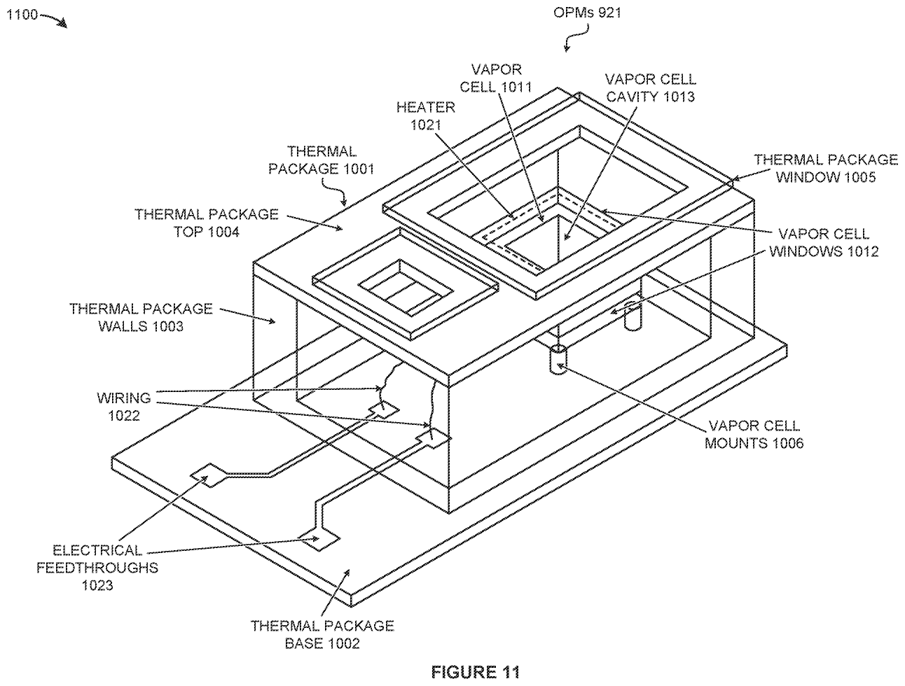

FIG. 11 illustrates view 1100. View 1100 is representative of an external perspective view of thermally packaged vapor cell 1011 in one of OPMs 921. Thermally packaged vapor cell 1011 illustrated in FIG. 11 is an example of atomic device 101 illustrated in FIGS. 1-3 and atomic device 401 illustrated in FIGS. 4-7, however devices 101 and 401 may differ. View 1100 illustrates thermal package 1001, vapor cell 1011, heater 1021, wiring 1022, and electrical feedthroughs 1023. Thermal package 1001 comprises thermal package base 1002, thermal package walls 1003, thermal package top 1004, thermal package window 1005, and vapor cell mount 1006. Thermal package 1001 is filled with xenon or krypton and is hermetically sealed. Vapor cell 1011 comprises vapor cell widows 1012 and vapor cell cavity 1013. Although not illustrated, a thermal package window is positioned on the bottom side of base 1002 to allow laser light emitted to pass through vapor cell 1011.

In some examples, thermal package base 1002 comprises a silicon wafer. Electrical feedthroughs 1023 are embedded onto the surface of base 1002. Wiring 1022 is soldered to contact points on electrical feedthroughs 1023 in the interior of package 1001. Thermal package walls 1003 comprise a rectangular borosilicate glass piece that surrounds cell 1011. Walls 1003 are anodically bonded to base 1002 to create a hermetic seal. In other examples, walls 1003 may be bonded to base 1002 with an adhesive, a solder, or another wafer-bonding method like laser-welding, thermocompression bonding, or direct bonding, but is not limited to these.

Vapor cell 1011 is representative of an atomic vapor cell. The walls of cell 1011 comprise silicon and cell windows 1012 comprise borosilicate glass hermetically bonded to the walls to form cell cavity 1013. A rubidium vapor is located within cell cavity 1013. Vapor cell 1011 is coupled to base 1002 via mount 1006. Cell 1011 may attach to mount 1006 using a physical connection (e.g., a snap socket) or an adhesive (e.g., an epoxy). Mount 1006 elevates cell 1001 above the surface of base 1002 to reduce conductive heat transfer between the surfaces of cell 1011 and base 1002. Mount 1006 comprises materials with low thermal conductivity like 3D printed plastic, polyamide, and the like. Mount 1006 is coupled to base 1002 by an adhesive. Although mount 1006 is illustrated as standoff posts, in other examples mount 1006 may comprise a different geometry.

Heater 1021 is a resistive heat element and comprises a metallic thin-film deposited onto the surface of one of vapor cell windows 1012. Heaters 1021 are coupled to external systems via wiring 1022 and electrical feedthroughs 1023. In some examples, heaters 1021 are omitted and heat is supplied to cell 1011 by light absorbed by the material of vapor cell 1011 or absorptive filters placed on the surface of vapor cell 1011. In some examples, heaters 1021 comprise a temperature sensor that contacts vapor cell 1011 to measure cell temperature. Thermal package top 1004 comprises a silicon wafer with view ports for thermal package window 1005. Top 1004 is anodically bonded to walls 1003 to create a hermetic seal. Window 1005 is anodically bonded to top 1004 over the view port. In other examples, top 1004 may be bonded to walls 1003 with an adhesive, a solder, or another wafer-bonding method like laser-welding, thermocompression bonding, or direct bonding, but is not limited to these. Different bonding techniques may be used to couple top 1004 to walls 1003 and walls 1003 to base 1002. Bonding base 1002, walls 1003, top 1004, and window 1005 to each other hermetically seals the filling gas (xenon or krypton) within the volume enclosed by thermal package 1001.

In operation, current is supplied to heaters 1021 via feedthroughs 1023 and wiring 1022. Heater 1021 receives the current and supplies heat to cell 1011. The xenon or krypton gas within thermal package 1001 inhibits conductive heat transfer between vapor cell 1011 and heater 1021 and the interior surfaces of thermal package 1001. Mount 1006 inhibits conductive heat transfer between vapor cell 1011 and the interior surfaces of thermal package 1001. The heat supplied by heater 1021 vaporizes the rubidium contained within cell cavity 1013 to pressurize cell 1011. Once pressurized, probe laser 922 emits a probe beam. The probe beam enters thermal package 1001 through window 1005. The probe beam then enters vapor cell cavity 1013 through the upper one of vapor cell windows 1012 and interacts with the rubidium atoms. The probe beam exits cell cavity 1013 through the lower one of vapor cell windows 1012 and exits thermal package 1011 through the window adhered to the bottom of base 1011. The probe beam is then detected by photodetector 924.

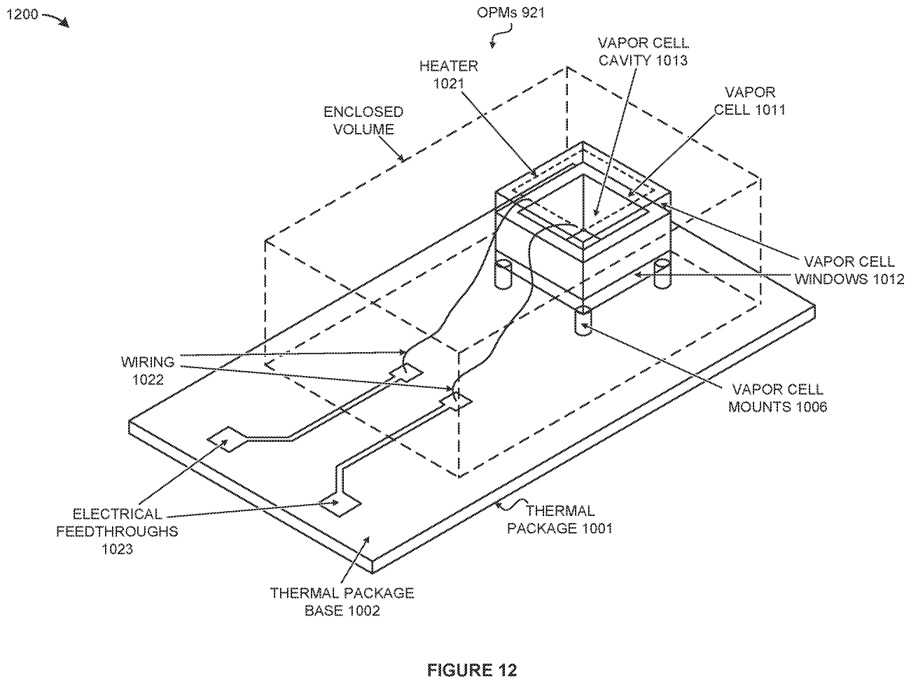

FIG. 12 illustrates view 1200. View 1200 is representative of an internal perspective view of thermally packaged vapor cell 1011 of one of OPMs 921. View 1200 illustrates thermal package base 1002, mount 1006, vapor cell 1011, vapor cell windows 1012, vapor cell cavity 1013, heater 1021, wiring 1022, and electrical feedthroughs 1023. The enclosed volume is filled with xenon or krypton gas. Mount 1006, vapor cell 1011, vapor cell windows 1012, vapor cell cavity 1013, heater 1021, wiring 1022, and a portion of electrical feedthroughs 1023 are positioned within the enclosed volume.

The illustrations of the embodiments described herein are intended to provide a general understanding of the structure of the various embodiments. While several implementations are described in connection with these illustrations of the embodiments, the disclosure is not limited to the implementations disclosed herein. On the contrary, the intent is to cover all alternatives, modifications, and equivalents. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Moreover, although specific embodiments have been illustrated and described herein, it should be appreciated that any subsequent arrangement designed to achieve the same or similar purpose may be substituted for the specific embodiments shown.

This disclosure is intended to cover any and all subsequent adaptations or variations of various embodiments. Combinations of the above embodiments, and other embodiments not specifically described herein, will be apparent to those of skill in the art upon reviewing the description. While, for purposes of simplicity of explanation, methods included herein may be in the form of a functional diagram, operational scenario or sequence, or flow diagram, and may be described as a series of acts, it is to be understood and appreciated that the methods are not limited by the order of acts, as some acts may, in accordance therewith, occur in a different order and/or concurrently with other acts from that shown and described herein. For example, those skilled in the art will understand and appreciate that a method could alternatively be represented as a series of interrelated states or events, such as in a state diagram. Moreover, not all acts illustrated in a methodology may be required for a novel implementation. Additionally, the illustrations are merely representational and may not be drawn to scale. Certain proportions within the illustrations may be exaggerated, while other proportions may be reduced. Accordingly, the disclosure and the figures are to be regarded as illustrative and not restrictive.

The various materials and manufacturing processes discussed herein are employed according to the descriptions above. However, it should be understood that the disclosures and enhancements herein are not limited to these materials and manufacturing processes, and can be applicable across a range of suitable materials and manufacturing processes. Thus, the descriptions and illustrations included herein depict specific implementations to teach those skilled in the art how to make and use the best options. For the purpose of teaching inventive principles, some conventional aspects have been simplified or omitted. Those skilled in the art will appreciate variations from these implementations that fall within the scope of this disclosure. Those skilled in the art will also appreciate that the features described above can be combined in various ways to form multiple implementations.

What is claimed is:

1. A thermally packaged atomic device, the atomic device comprising:
an atomic vapor cell;
at least one heater configured to heat the atomic vapor cell; and
an enclosure filled with a gas selected for comprising a thermal conductivity less than air that surrounds the atomic vapor cell with the gas.

2. The thermally packaged atomic device of claim 1 wherein the gas comprises xenon.

3. The thermally packaged atomic device of claim 1 wherein the gas comprises krypton.

4. The thermally packaged atomic device of claim 1 wherein the gas comprises argon.

5. The thermally packaged atomic device of claim 1 further comprising a vapor cell mount to couple the atomic vapor cell to the enclosure.

6. The thermally packaged atomic device of claim 1 wherein the enclosure is sealed with an adhesive.

7. The thermally packaged atomic device of claim 1 wherein the enclosure is sealed with a laser bond.

8. The thermally packaged atomic device of claim 1 wherein the enclosure is sealed with solder.

9. The thermally packaged atomic device of claim 1 wherein the enclosure is sealed with an anodic bond.

10. The thermally packaged atomic device of claim 1 further comprising electrical feedthroughs; and wherein:
the electrical feedthroughs are embedded into the surface of the enclosure and provide power to the at least one heater.

11. The thermally packaged atomic device of claim 1 further comprising a temperature sensor coupled to the atomic vapor cell; and wherein:
the temperature sensor is configured to sense a temperature of the atomic vapor cell.

12. The thermally packaged atomic device of claim 1 wherein the atomic vapor cell comprises alkali atoms.

13. The thermally packaged atomic device of claim 1 wherein the atomic device comprises a magnetic field sensor.

14. The thermally packaged atomic device of claim 1 wherein the atomic device comprises an atomic frequency reference.

15. The thermally packaged atomic device of claim 1 wherein the atomic device comprises a gyroscope.

16. The thermally packaged atomic device of claim 1 wherein the atomic device comprises an electrometry sensor.

17. A method of manufacturing a thermally packaged atomic device, the method of manufacturing comprising:
mounting an atomic vapor cell to an enclosure base;
placing enclosure walls on the enclosure base to surround the atomic vapor cell;
clamping the enclosure walls to the enclosure base;
bonding the enclosure walls to the enclosure base at an interface between the enclosure walls and the enclosure base;
backfilling a surrounding environment of the atomic vapor cell with a gas selected for comprising a thermal conductivity less than air and placing an enclosure top on the enclosure walls to create an enclosed volume that surrounds the atomic vapor cell with the gas;
clamping the enclosure top to the enclosure walls; and
bonding the enclosure top to the enclosure walls at an interface between the enclosure top and the enclosure walls.

18. The method of manufacturing of claim 17 wherein backfilling the surrounding environment with the gas selected for comprising a thermal conductivity less than air comprises backfilling the surrounding environment with xenon.

19. The method of manufacturing of claim 15 wherein backfilling the surrounding environment with the gas selected for comprising a thermal conductivity less than air comprises backfilling the surrounding environment with krypton.

20. The method of manufacturing of claim 17 further comprising:
embedding electrical feedthroughs onto an upper surface of the enclosure base; and
wiring the electrical feedthroughs to a heater mounted to the atomic vapor cell.

21. The method of manufacturing of claim 17 wherein:
bonding the enclosure walls to the enclosure base at the interface between the enclosure walls and the enclosure base comprising laser welding the enclosure walls to the enclosure base at the interface between the enclosure walls and the enclosure base; and
bonding the enclosure top to the enclosure walls at the interface between the enclosure top and the enclosure walls comprises laser welding the enclosure top to the enclosure walls at the interface between the enclosure top and the enclosure walls.

22. The method of manufacturing of claim 17 wherein:
bonding the enclosure walls to the enclosure base at the interface between the enclosure walls and the enclosure base comprising soldering the enclosure walls to the enclosure base at the interface between the enclosure walls and the enclosure base; and
bonding the enclosure top to the enclosure walls at the interface between the enclosure top and the enclosure walls comprises soldering the enclosure top to the enclosure walls at the interface between the enclosure top and the enclosure walls.
